(19) United States
(12) Patent Application Publication
(10) Pub. No.: US 2014/0213548 A1
(43) Pub. Date: Jul. 31, 2014

(54) SYSTEM AND METHOD FOR HEMOSTATIC WOUND DRESSING

(71) Applicant: New Jersey Institute of Technology, Newark, NJ (US)

(72) Inventors: George J. Ulsh, Mullica Hill, NJ (US); Jennifer W. Moy, North Cladwell, NJ (US); Maxwell A. McDermott, Clinton, NJ (US); Dung T. Le, Mullical Hill, NJ (US); George Colling, Maplewood (NJ); Jessica Cardenas Turner, North Brunswick, NJ (US)

(73) Assignee: New Jersey Institute of Technology, Newark, NJ (US)

(21) Appl. No.: 14/165,147

(22) Filed: Jan. 27, 2014

Related U.S. Application Data
(60) Provisional application No. 61/757,195, filed on Jan. 27, 2013.

(51) Int. Cl. A61L 15/44; A61L 15/20

(52) U.S. Cl. CPC A61L 15/44; A61L 15/20
USPC 514/55

(57) ABSTRACT

The present invention relates a novel hemostatic wound dressing, preferably comprising chitosan and an oxygen carrier such as a perfluorocarbon, and methods to treat hemorrhaging wounds and MRSA infections. The present invention helps with coagulation/clot formation as well as providing oxygen to the wound, all while being cost effective and competitive with current hemostatic dressings.

This application claims the benefit of US Provisional Application No. 61/757,195, filed Jan. 27, 2013.

The present invention relates a novel hemostatic wound dressing. The present invention helps with coagulation/clot formation as well as providing oxygen to the wound, all while being cost effective and competitive with current hemostatic dressings.

### BACKGROUND OF THE INVENTION

Nearly a quarter of the 4,596 combat deaths in Iraq and Afghanistan between 2001 and 2011 were “potentially survivable”. Uncontrolled blood loss was the leading cause of death in 90% of the potentially survivable battlefield cases and in 80% of those who died in a military treatment facility. Many of these of the deaths occurred before the injured reached a medical facility: of the troops who suffered mortal wounds on the battlefield, about one third died instantly and two thirds succumbed before arriving at a treatment center. There is little that can be done in the field to control “bleed-outs”, especially those caused by groin or neck wounds where there is blood loss caused by major arterial injuries.

Thus there remains a need for more effective treatment of battlefield wounds to enable survival until a treatment center can be reached.

The most common and deadly hospital acquired infection is Methicillin-resistant Staphylococcus aureus (MRSA). MRSA is highly adaptable and very resistant to antibiotics. Although various MRSA prevention protocols have been developed, there remains a need for preventing the establishment of a MRSA infection in the first place.

Thus there remains a need for an effective wound dressing that can provide a non-toxic protective barrier over a damaged area or surgical with greatly enhanced bacterial and fungal resistance that can be used in hospitals to protect against hospital borne infection.

### SUMMARY OF THE INVENTION

The present invention creates a cost effective, chitosan based wound dressing material with inherent microbiocidal properties and oxygen release that can be used in a wide range of applications to create a non-toxic protective barrier over the damaged area with greatly enhanced bacterial and fungal resistance.

The present invention helps with coagulation/clot formation as well as providing oxygen to the wound, all while being cost effective and competitive with current hemostatic dressings.

In certain embodiments of the present invention the wound dressing is comprised of a micro/nano sized fibrous mat made of chitosan that is doped with perfluorocarbons which act as an oxygen carrier.

Another embodiment of the invention relates to a method of treating a hemorrhaging wound by packing the wound with the hemostatic dressing of the present invention in order to stop the bleeding. Under this methodology of packing a hemorrhaging wound, the chitosan rapidly absorbs the blood in the wound bed and forms a gelatinous clot that fills the empty void of the wound. The gelatinous clot filling this void in the tissue applies pressure to the damaged vasculature, which prevents further bleeding. Chitosan also activates the clotting cascade and causes the agglutination of red blood cells, thus accelerating coagulation by influencing the activation of platelets. The perfluorocarbon (PFC) acts as an oxygen carrier to release oxygen into the wound bed to help facilitate the wound healing process.

### BRIEF DESCRIPTION OF THE DRAWINGS

So that those having ordinary skill in the art will have a better understanding of how to make and use the disclosed systems and methods, reference is made to the accompanying figures wherein:

FIG. 1 shows a SEM Image of electrospun chitosan nano-fiber mat at 50,000× original magnification;

FIG. 2 shows a chitosan nano-fiber mat;

FIG. 3 shows chitosan with varying weight percent (wt %), which are 1 wt %, 2 wt %, 3 wt %, 5 wt %, and 7 wt % chitosan from left to right) in 90 volume percent (v/v) % acetic acid;

FIG. 4 shows a thin section of a chitosan mat;

FIG. 5 shows the process of electrospinning chitosan solution;

FIG. 6 shows chitosan nanofibers on the collector after electrospinning chitosan solution (7% (w/v) chitosan in Trifluoroacetic acid solution;

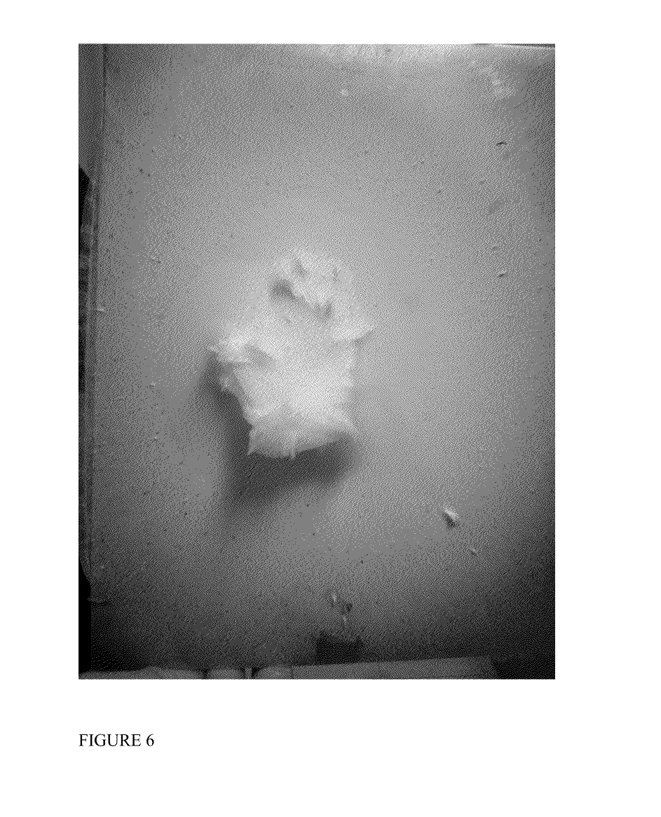

FIG. 7 shows an unprocessed chitosan nanofiber mat; and

FIG. 8 shows the application of porcine blood onto the chitosan mat (a) and a blood clot formation when the chitosan mat has been exposed to porcine blood after five minutes (b).

### DETAILED DESCRIPTION OF THE INVENTION

This invention relates to a hemostatic wound dressing which comprises chitosan and an oxygen carrier.

In an embodiment of the invention the chitosan is electrospun into a nano-fiber mat. In another embodiment the chitosan may be force spun by means known in the art.

In another embodiment of the invention, the wound dressing is characterized as a packing gauze, which can be packed into a wound. In such an embodiment, the wound dressing comprises chitosan and an oxygen carrier.

In another embodiment of the invention, the wound dressing is characterized as a topical bandage which comprises chitosan, an oxygen carrier and an adhesive backing.

Another embodiment relates to a “smart bandage” which comprises chitosan, an oxygen carrier and a biodegradable polymer such as zein.

The oxygen carrier of the invention can be any compatible non-toxic oxygen carrying molecule. A preferred class of compounds are the perfluorocarbons, including but not limited to perfluorotributylamine (PFTBA), perfluorodecalin, bis(perfluorohexyl)ethane, perfluorotripropylamine, perfluorooctylbromide and bis(perfluorobutyl)ethane). More preferred is PFTBA.

Another embodiment of the invention relates to a method of treating a hemorrhaging wound by packing the wound with the wound dressing of the invention.

Yet another embodiment of the invention relates to a method of preventing a MRSA infection by bandaging the wound with a wound dressing of the invention.

Chitosan, utilized in multiple embodiments of the present invention, is effective against bacterial and fungi because the cationic polymer targets bacterial cell membranes of both gram positive and gram negative bacteria. In the present invention, chitosan swells to form a gelatinous clot. In the present invention, an oxygen carrier, for example, a perfluorocarbon, provides oxygen that specialized enzymes can bind to in order to convert the oxygen into reactive oxygen species. Reactive oxygen species are known to act as cellular messengers within wound healing. Additionally, the reactive oxygen species are capable of destroying bacteria and are naturally used within the body by the immune system. By providing oxygen to the wound bed during the initial stage of wound healing, the immune cells could utilize the oxygen in order to destroy the present bacteria before an infection could develop. After clearing the infection, the body can utilize the remaining oxygen for repairing and remodeling of the wound bed. Embodiments of the present invention rapidly absorb the blood in the wound bed and form a gelatinous clot that fills the empty void in the wound. By the gelatinous clot filling this void in the tissue, pressure is applied to the damaged vasculature which prevents further bleeding which also causes the agglutination of red blood cells, accelerates coagulation in vivo by influencing the activation of platelets.

In one embodiment of the present invention, the PFTBA releases oxygen into the wound bed which oxygenates the damaged tissue and aids in the formation of oxygen radicals. Over time, the chitosan in the present invention will be absorbed by the body and converted into sugar, while the perfluorocarbon (PFC) is expelled from the body.

Between about 01 wt % and about 5 wt % and more preferably between about 1 wt % and about 2 wt % allows for electrospinning in the present embodiment.

The present invention, in one embodiment, is meant to replace existing traditional cotton gauzes and current hemostatic gauzes, used for traumatic injuries. One method of use of the present invention involves taking the novel hemostatic material and packing it in a wound so that it stops bleeding by rapidly swelling and forming a gelatinous clot. The chitosan in said present embodiment eventually breaks down into sugars and is reabsorbed into the body.

Another method of use for the present invention is to add an adhesive material to one side of the novel hemostatic material so that the hemostatic material can be held in place in the same manner as existing bandages.

Yet another embodiment of the invention contemplates the formation of wound dressings that have a biodegradable membrane on a portion of the hemostatic material, for example a smart bandage comprising chitosan, an oxygen carrier and a biodegradable polymer such as zein.

Zein as a material has been reported to have natural microbial resistance, as well as film-forming properties. In addition, it is plant derived which makes it renewable, abundant and biodegradable.

Zein has been observed to easily form electrospun fibers. The stability of the electrospun zein fibers in aqueous environment was improved using cross linkers. After electrospinning of the crosslinked solution, the fibers were cured at 150° C. for 2 hour. Fibers were immersed separately in deionized water and PBS. It was observed that after 1 hour of curing the fibers reached a certain level of cross-linking and did not dissolve. The fiber morphology for the uncrosslinked and 5% was not retained, they swelled and collapsed into a film after 1 and 2 hours of curing. Such constructs should be useful in preparing smart bandages of the invention.

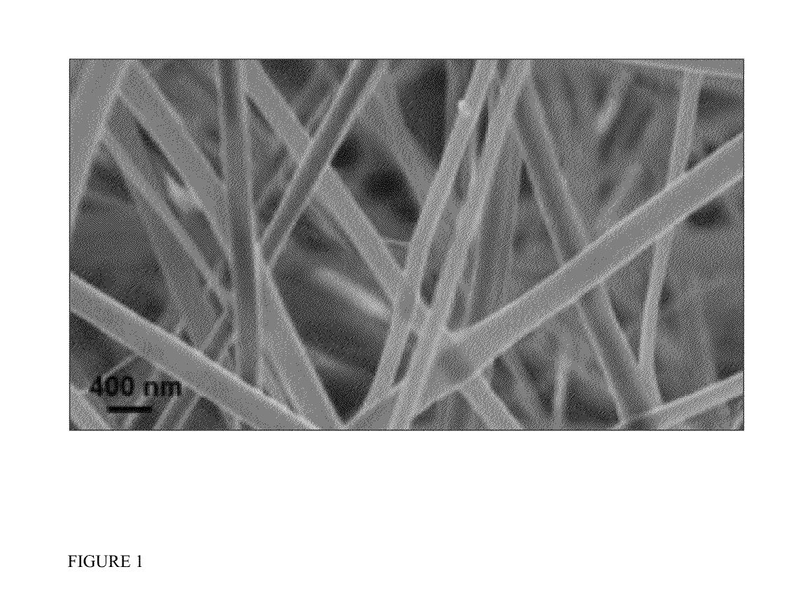

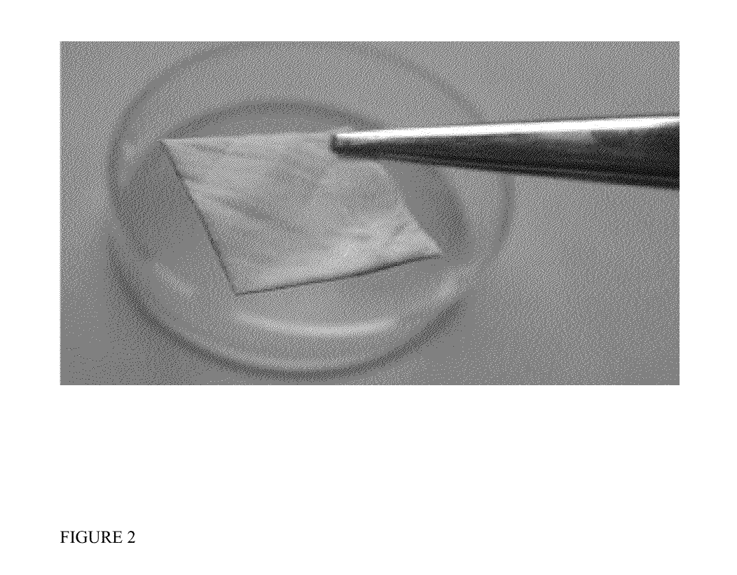

In certain embodiments of the present invention the chitosan used is electrospun chitosan. Chitosan is naturally hemostatic, microbiocidal, and bioresorbable lending itself to be a perfect wound healing material. The chitosan causes increased permeability and the rupture of bacterial and fungal membranes. Chitosan has been electrospun in certain embodiments of the present invention into a micro/nano-fiber mat because it allowed maximization of surface area for adhesion and in turn, clotting. FIG. 1 shows a SEM Image of electrospun chitosan nano-fiber mat at 50,000× original magnification, while FIG. 2 shows a chitosan nano-fiber mat of the invention.

In conjunction with the electrospun mats of chitosan, the present invention in certain embodiments contains an oxygen carrier. One example of a class of oxygen carriers that are utilized in the present invention is PFC (i.e. perfluorotributylamine PFTBA). However, the present invention is not limited to this particular class or example of oxygen carriers. Oxygen is extremely soluble in PFCs and can completely release withheld oxygen in the presence of a concentration gradient. By diffusing oxygen into the wound embodiments of the present invention increase cell viability around the damaged tissue. Secondly, the negative oxygen ion elicits an immune response furthering functionality. Both chitosan and PFTBA have been used extensively in other applications and are approved by the FDA.

It is also contemplated within the invention that a wound dressing

Embodiments of the present invention initiate clot formation rapidly and the whole dressing is completely reabsorbed or expelled by the body. The present invention oxygenates cells and tissues at the damage site and surrounding areas. The present invention allows for dramatic reduction in secondary infections with active ingredients that are approved by FDA.

### Experimental

### Preparation of Chitosan in Acetic Acid

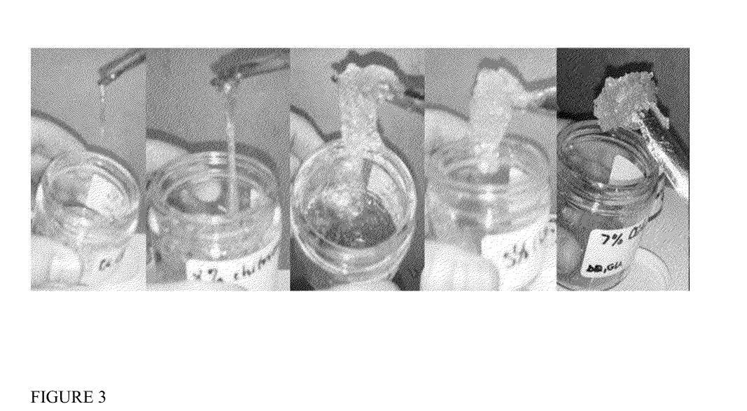

An acid solution was prepared by mixing acetic acid (Sigma Aldrich, St. Louis, Mo.) with deionized water in a 9:1 volume ratio. The resulting aqueous acid solution was poured into varying amounts of chitosan (medium molecular weight; Sigma Aldrich). All the chitosan solution was stirred at 1200 rpm for 24 hours. The amount of chitosan in the solution determines the viscosity of the solution, as seen in FIG. 3.

### Preparation of Chitosan in Trifluoroacetic Acid

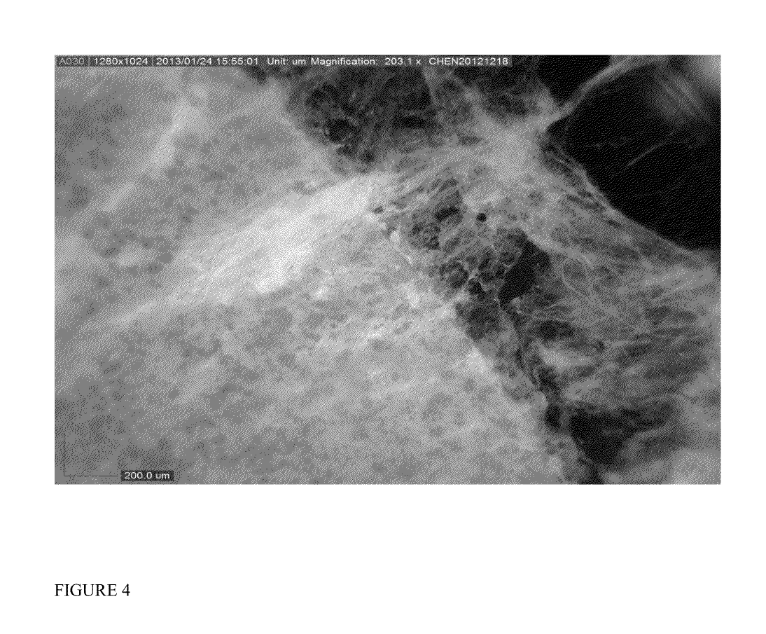

In another exemplary embodiment of the present invention, trifluoroacetic acid was used instead of acetic acid. By using a stronger acid, the present embodiment was able to dissolve a much higher wt % of chitosan than the previous embodiment. A thin section of the mat created utilizing the present embodiment can be seen in FIG. 4. The scale of FIG. 4 is 200 μm and was magnified 203.1× times using a digital microscope.

A 7% (w/v) chitosan solution was prepared by dissolving chitosan (Sigma Aldrich) in an acid solution. The acid solution contained 80% (v/v) trifluoroacetic acid (Fisher Chemicals, Pittsburgh, Pa.) and 20% (v/v) methylene chloride (Fisher Chemicals). The chitosan solution was stirred at 60° C. overnight.

### Electrospinning Chitosan in Trifluoroacetic Acid

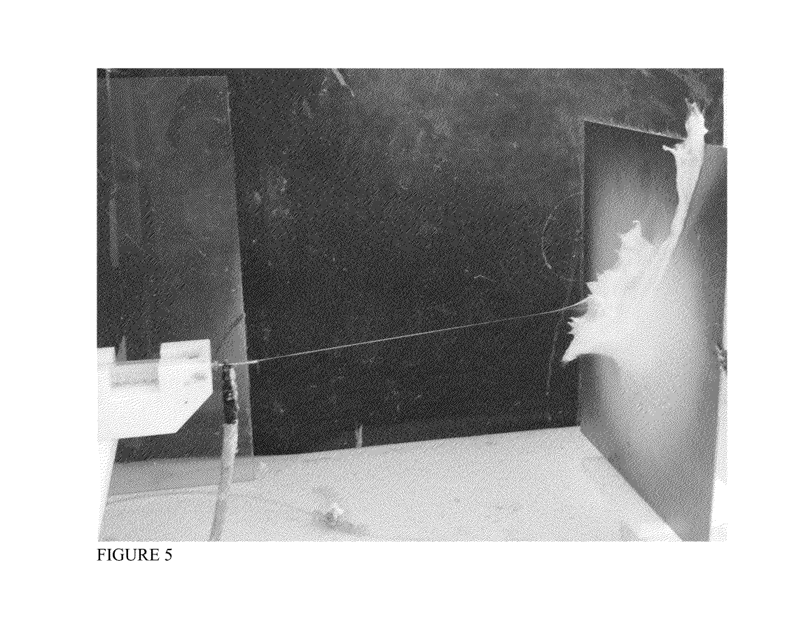

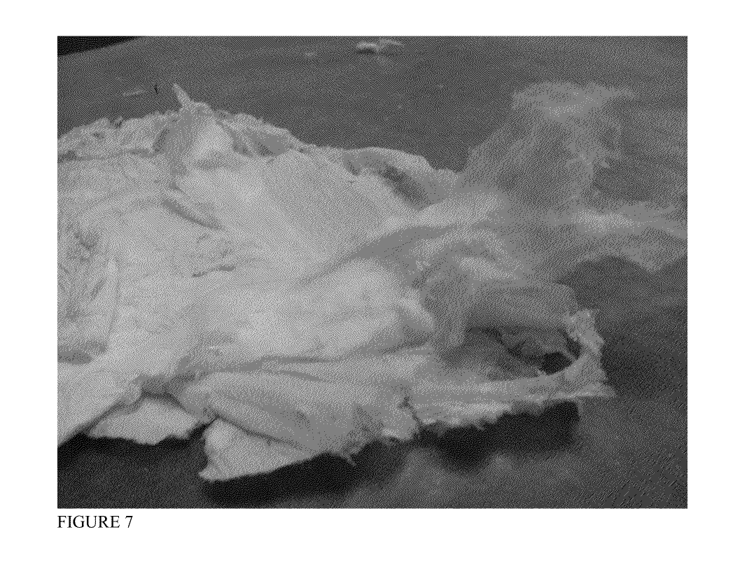

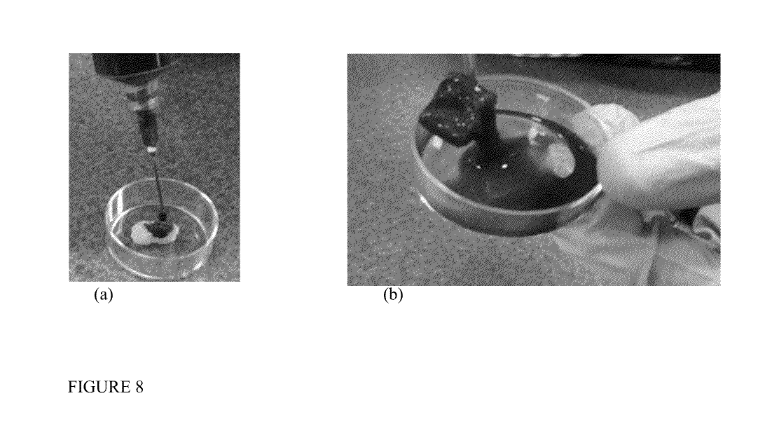

The chitosan-trifluoroacetic acid solution was fed into a 10 mL disposable syringe. 20-gauge needle was used. A DC voltage of 50 kV was applied between the tip of the needle and the collector that was placed 30 cm away from the needle as shown in FIG. 5. The solution was pumped at a rate of 2 mL per hour. The electrospinning process was performed at a temperature of 20-23° C. with the humidity of 30-33%. A humidifier was used to control the humidity during electrospinning process since humidity was one of the parameters that greatly affect the electrospinnability. FIG. 7 shows the chitosan nanofibers on the collector after electrospinning chitosan solution (7% (w/v) chitosan in Trifluoroacetic acid solution. FIG. 8 shows an unprocessed chitosan nanofiber mat.

Preparation of Chitosan in Trifluoroacetic Acid with an Addition of Perfluorotributylamine

A 7% (w/v) chitosan solution was prepared by dissolving chitosan (Sigma Aldrich) in an acid solution. The acid solution contained 80% (v/v) trifluoroacetic acid (Fisher Chemicals) and 20% (v/v) methylene chloride (Fisher Chemicals). The chitosan solution was stirred for 45 minutes at 60° C. An oxygenated PFTBA (Sigma Aldrich) was prepared by bubbling oxygen from an air gas tank in a PFTBA liquid. After the chitosan solution was stirred for 45 minutes, the oxygenated PFTBA was added in the chitosan solution in 1:10 ratio. The final solution was stirred for 2 minutes at 60° C.

### Electrospinning Chitosan in Trifluoroacetic Acid and Perfluorotributylamine

The chitosan-trifluoroacetic acid-PFTBA solution was fed into a 10 mL disposable syringe. 20-gauge needle was used. A DC voltage of 50 kV was applied between the tip of the needle and the collector that was placed 15cm away from the needle. The solution was pumped at a rate of 2 mL per hour. During the electrospinning process, the final solution within the syringe was heated to 60° C. while the room temperature was 20-23° C. and the humidity was 30-33%. The

### Oxygen Release

The oxygen releasing experiment was performed in an inflatable glove chamber (Sigma Aldrich). The glove chamber was inflated with helium gas. An Optical Dissolved Oxygen Probe (Vernier, Beaverton, Oreg.) was used to measure the amount of dissolved oxygen in 20 mL of deionized water. Helium was pumped into the 20 mL water for 30 minutes to remove the oxygen from the water, while the Oxygen Probe was continuously collecting date within the water. After the deionized water became deoxygenated, the chitosan nanofibers were placed in the water and the amount of dissolved oxygen was determined.

For electrospinning, the chitosan-trifluoroacetic acid-PFTBA solution had a volume of 4 mL. The amount of PFTBA in that solution was 0.4 mL. The fibrous mat collected from electrospinning 4 mL of solution had a weight of 0.1534 g. After collecting the mat, the release rate of oxygen was tested. The initial concentration of dissolved oxygen in deionized water was 8.53±0.02 mg/L. After helium gas was pumped in the water, the concentration of dissolved oxygen decreased to 1.13±0.02 mg/L. The concentration of dissolved oxygen increased to 2.62±0.03 mg/L. For a mat weighing 0.1534 g, it released 1.49 mg/L of oxygen

### Blood Clotting Simulation

A segment of the electrospun chitosan mat was cut into a 1 cm by 1 cm square and placed into a petri dish. Fresh porcine blood obtained from a local butcher was loaded into a syringe. The porcine blood was injected over the chitosan mat so that the blood would fully coat the mat and the surface of the petri dish. After five minutes, excess blood was removed from the mat by lifting the chitosan mat with a pair of tweezers and tipping the petri dish. A blood clot had formed on the mat.

Although the systems and methods of the present disclosure have been described with reference to exemplary embodiments thereof, the present disclosure is not limited thereby. Indeed, the exemplary embodiments are implementations of the disclosed systems and methods are provided for illustrative and non-limitative purposes. Changes, modifications, enhancements and/or refinements to the disclosed systems and methods may be made without departing from the spirit or scope of the present disclosure. Accordingly, such changes, modifications, enhancements and/or refinements are encompassed within the scope of the present invention.

1. A hemostatic wound dressing which comprises chitosan and an oxygen carrier.

2. The wound dressing of claim 1 wherein the chitosan is electrospun into a nano-fiber mat.

3. The wound dressing of claim 1 wherein the chitosan is force spun into a nano-fiber mat.

4. The wound dressing of claim 1 which is a packing gauze.

5. The wound dressing of claim 4 which comprises chitosan and an oxygen carrier.

6. The wound dressing of claim 1 which is an orthopedic bandage.

7. The wound dressing of claim 6 which comprises chitosan, an oxygen carrier and an adhesive backing.

8. The wound dressing of claim 1 wherein the oxygen carrier is a perfluorocarbon.

9. The wound dressing of claim 8 wherein the perfluorocarbon is selected from the group of perfluorotributylamine (PFTBA), perfluorodecalin, bis(perfluorohexyl)ethane, perfluorotripropylamine, perfluorooctylbromide and bis(perfluorobutyl)ethane).

10. The wound dressing of claim 9 wherein the perfluorocarbon is PFTBA.

11. A method of treating a hemorrhaging wound which comprises packing the wound with the wound dressing comprising chitosan and an oxygen carrier.

12. A method of preventing a MRSA infection by bandaging the wound with a wound dressing of the invention.
